(19) United States
(12) Patent Application Publication
(10) Pub. No.: US 2013/0203613 A1
(43) Pub. Date: Aug. 8, 2013

(54) DEVICE AND METHOD FOR THE VERIFICATION AND QUANTITATIVE ANALYSIS OF ANALYTES, PARTICULARLY MYCOTOXINS

(71) Applicants: Jens Burmeister, Leverkusen (DE); Ingmar Dorn, Koln (DE); Viktoria Bazilyanska, Koln (DE); Ulrich Raczek, Leverkusen (DE)

(72) Inventors: Jens Burmeister, Leverkusen (DE); Ingmar Dorn, Koln (DE); Viktoria Bazilyanska, Koln (DE); Ulrich Raczek, Leverkusen (DE)

(73) Assignee: BAYER CROPSCIENCE AG, Monheim (DE)

(21) Appl. No.: 13/263,373

(22) PCT Filed: Mar. 26, 2010
(86) PCT No.: PCT/EP10/01924

§ 371 Date: Nov. 14, 2011

(30) Foreign Application Priority Data
Apr. 9, 2009 (EP) 09157714.8

(51) Int. Cl. G01N 33/53

(52) U.S. Cl. CPC G01N 33/5304; G01N 33/5308
USPC 506/9; 506/18

(57) ABSTRACT

The present invention relates to a device and a method for the verification and quantitative analysis of analytes and their application for the verification and quantitative analysis of mycotoxins.

This application is a 371 of PCT/EP2010/001924, filed Mar. 26, 2010, which claims foreign priority benefit under 35 U.S.C. §119 of the European Patent Application No. 09157714.8 filed Apr. 9, 2009, the disclosures of which are incorporated herein by reference.

The present invention relates to a device and a method for the verification and quantitative analysis of analytes and their use for the verification and quantitative analysis of mycotoxins.

In biochemistry and medicine, analyses are frequently based on the detection of an interaction between a molecule present in a known amount and position (the molecular probe) and an unknown molecule to be detected (the molecular target molecule).

In order to detect an interaction, a probe which is usually fixed to a support and is contacted with a target molecule present in a sample solution and incubated under defined conditions. As a consequence of said incubation, a specific interaction takes place between probe and target, which can be detected in various ways. Detection is based on the fact that a target molecule can form a specific bond only with certain probe molecules. Said bond is distinctly more stable than the bond of target molecules to probes which are not specific for the target molecule. The target molecules which have not been bound specifically can be removed by washing, while the probes hold onto the specifically bound target molecules.

In modern assays, a multiplicity of probes is deposited in the form of a substance library by way of a matrix (array) on a support in such a way that a sample can be analyzed in parallel on a plurality of probes at the same time (D. J. Lockhart, E. A. Winzeler, Genomics, gene expression and DNA arrays; Nature 2000, 405, 827-836).

The specific interaction between a target and its probe can then be detected on the basis of a “marker” by a multiplicity of methods which normally depend on the type of marker which has been introduced before, during or after said interaction of the target molecule with the probes. Typically, such markers are fluorescent groups, and specific target-probe interactions can therefore be read out in a fluorescence-optical manner with high spatial resolution and with little effort compared to other conventional detection methods, especially mass-sensitive methods (A. Marshall, J. Hodgson, DNA Chips: An array of possibilities, Nature Biotechnology 1998, 16, 27-31; G. Ramsay, DNA Chips: State of the art, Nature Biotechnology 1998, 16, 40-44).

Particularly advantageous in this context is the use of an evanescent-field biochip as support for the probe molecules. An evanescent-field biochip comprises an optical waveguide which can be used for detecting changes of the optical properties of a medium bordering the wave-guiding layer. When light is transported by way of a guided mode in the wave-guiding layer, the field of light does not drop off abruptly at the medium/waveguide interface but decreases exponentially in the “detection medium” adjacent to the waveguide. This exponentially decreasing field of light is referred to as evanescent field. A change in the optical properties of the medium bordering the waveguide within the evanescent field can be detected by a suitable measurement setup.

It is therefore possible to carry out detection on the specific binding of target molecules to probes immobilized on the waveguide via the changing optical properties of the waveguide/immobilized material boundary layer.

Preference is given to detecting a fluorescence signal in the evanescent field. The fluorescently labeled probe/target molecule binding pair is excited by an evanescent field. An example of an evanescent field biochip is given in U.S. Pat. No. 5,959,292.

Depending on a substance library of probes immobilized on the support and on the chemical nature of the target molecules, it is possible, on the basis of this assay principle, to study, for example, interactions between nucleic acids and nucleic acids, between proteins and proteins, antibodies and antigens, and between nucleic acids and proteins.

In order to facilitate a practical rapid detection method, it has been attempted for some years now to miniaturize chemo- and/or biosensor equipment and to complete nearly all reagents that are required for the qualitative and/or quantitative determination of a sample in a “cartridge”, ready to use. More specifically, microfluidics technology is employed with the goal of making available inexpensive, storable and easy-to-operate disposable cassettes which can deliver reproducible results in real time.

Regarding the storability and transportability of cartridges, the prior art makes use in particular of the dry assay technology in which all reagents are provided in the dry state in the cassette, where appropriate in separate chambers. The sample fluid is usually transported from one chamber to the next by means of microfluidic channels.

For example, WO 2005/088300 describes an integrated microfluidic cartridge for blood analysis, which consists of a lower and an upper body part. Both elements are structured with chambers and channels which are closed by combining the two parts. The test cassette has one or more pretreatment elements (pretreatment chamber) for preparing a sample, one or more multilayered dry assay elements (detection chamber) for recognizing one or more target molecules of a sample fluid, and one or more channels (average ≦3 mm) connecting the pretreatment elements with the multilayered dry assay elements. The pretreatment elements are in particular filter elements or elements having porous properties in the form of a channel or a (micro/nano) pad which may or may not bear dry reagents. The sample is first conducted through the pretreatment elements, then into the multilayered dry assay element. The multilayered dry assay recognition element has at least one functional layer bearing probes for a qualitative and quantitative assay of the target molecules in a dry and stable form. This reagent layer consists of a water-absorbing layer in which excitable probes are distributed fairly regularly in a hydrophilic polymeric binding material (gelatin, agarose, etc.). Detection is carried out by way of reflection photometry through a light-transparent window, by illuminating a detection layer in the multilayered dry assay element, in which layer the optically excitable fluid from the recognition reaction is diffused. The sample is transported by employing capillary forces or pressure. The disadvantage of this device is the complexity of the design of the multilayered dry assay element and the suboptimal mixing of the analyte with the detection reagents. Moreover, a precise time control of the individual reaction steps, in particular of the volumes and incubation times, is not possible, and the test results are therefore not reproducible quantitatively. Referencing is not described.

The lateral flow assay (LFA) technology has also been known for biochemical analysis for many years now. Lateral flow assays (LFAs) utilize the effect of the antibody-antigen reaction. In addition, the sample (solution) to be analyzed is pulled across the sensor surface by capillary forces. To detect analytes by means of LFAs, for example, a direct, competitive immunoassay may be performed on a nitrocellulose strip, with the sample to be analyzed being pulled through the entire nitrocellulose strip due to capillary forces. The zone in which the anti-analyte antibody has been immobilized is used as detection zone for the strip assay. An example of an LFA for detecting mycotoxins (e.g. deoxynivalenol) is the reveal assay (test cassette) from Neogen, Lansing, MI, USA with the corresponding AccuScan reader. The cartridge is inserted into the reader, and the instrument records an image of the results area of the strip assay. The reader interprets the results image, and an evaluation is made, when a line has been recognized. The instrument eliminates the subjectivity of interpretation and provides objective, comprehensible documentation of the test result. The assay described can be carried out easily and relatively quickly and does not need any complex readout instruments. Disadvantageously, the method allows only a qualitative or at most semi-quantitative mycotoxin detection.

WO 2007/079893 describes a method for rapidly detecting mycotoxin, which comprises applying a supported substance library of immobilized binding partners for mycotoxins and/or probes for mycotoxins in spatially separated measurement areas to the surface of a thin-layer waveguide, contacting a sample containing mycotoxin and probes of said mycotoxin with the immobilized binding partners, and detecting the reaction of said immobilized binding partners with the mycotoxins and/or recognition elements of said mycotoxins on the basis of a signal change in the evanescent field, i.e. at the interface to the waveguide. Particularly advantageously, the method can, to a limited extent or even completely, dispense with washing off fluorescently labeled binding partners or a sample or solution containing labeled binding partners prior to detection of a signal. This can both save time during analysis and simplify the procedure, since providing washing solutions can also be dispensed with. The signal intensity is determined on the basis of a recorded image of the assay by means of a suitable software, as is the calculation of the amount of mycotoxins present in the sample. However, the prior art has disclosed that a suitable referencing method is advantageous for the reliability of the quantitative analysis. WO 2007/079893 does not describe such a referencing method.

The prior art describes utilization of one or more measurement areas for calibration of an assay. For example WO 01/13096 uses measurement areas for referencing identical chemical or optical parameters (for example the intensity of the locally available excitation light) in a plurality of sample containers distributed across the sensor platform so as to enable the local distribution of said parameters on the sensor platform to be determined. The number and position of measurement areas for referencing in the above-mentioned arrangement of measurement areas is random.

EP-A 0 093 613 describes a method for calibrating an assay for quantifying a target molecule in a sample fluid by means of a sensor based on fluorescence excitation in the evanescent field of an optical waveguide, which sensor has a first measurement area (measuring area) for specifically binding a first label which is used in an amount that is a function of the presence of an analyte in the sample, and a second measurement area (calibrating area) for binding a second label, the binding of which is not influenced by the presence of the analyte in the sample. The measuring areas and calibrating areas make use of different binding pairs which are of a similar nature, however. The quantity of the second label in the calibrating area during the assay gives a signal value for a predefined concentration of the analyte within a concentration area. Both measurement areas are placed close to one another, on the same basic structure, in order to minimize differences caused by possible local variations of the sensor. The signal value of the measuring area is divided by the signal value of the calibrating area placed closely thereto, in order to correct the nonspecific effects of the sensor on the signal. The design of the sensor and the direction of the excitation beam are not defined in any detail.

WO 2004/023142 describes a method for calibrating an assay for quantifying a target molecule in a sample fluid by means of a sensor based on fluorescence excitation in the evanescent field of an optical waveguide, onto which sensor recognition elements and reference molecules (Cy5-BSA, BSA=bovine serum albumin) have been spotted in measurement spots and reference spots, respectively, in separate parallel alternating microarrays orthogonally to the direction of propagation of the excitation light conducted in the evanescent-field sensor platform. To reference the signal intensity of each measurement spot, the net signal intensity of said measurement spot is divided by the average of the net signal intensities of the adjacent reference spots of the same row, arranged in the direction of propagation of the excitation light. This referencing compensates for the local differences of the available excitation light intensity orthogonally to the direction of light propagation, both within each microarray and between various microarrays.

When using the methods of referencing described in the prior art, they turned out to be unsuitable for referencing the assays in a fluidic system. It turned out, when using spotted, fluorescent proteins as reference, that only those fluctuations of the system can be compensated for which occur at the level of the sensor, such as, for example, attenuation of the fluorescence light or fluctuations in the spotting of the arrays.

From the prior art, it was an object to provide an inexpensive, storable and easy to operate means for the quantitative analysis of analytes, in particular mycotoxins, by means of a substance library of immobilized binding partners in spatially separated measurement areas (immunoassay) on a thin-film waveguide (PWG biochip, PWG=planar waveguide) support. A further object of the present invention is that of absolute determination, i.e. referencing, of the signal generated.

This object is achieved according to the invention by a microfluidic cartridge for the qualitative and/or quantitative analysis of analytes, in particular of mycotoxins, which includes, in a dry form, all reagents required for carrying out the assay. The cartridge of the invention has a structured body into which cavities connected to one another by channels have been inserted. According to the invention, the cartridge has at least one inlet for introducing a mycotoxin-containing sample fluid, at least one reagent chamber and at least one detection chamber. The reagent chamber accommodates, in a dry form, one or more labeled mycotoxin probes to react with the mycotoxins from the sample fluid and labeled referencing probes to react with a referencing antigen. The bottom of the detection chamber consists of a thin-film waveguide (PWG biochip) comprising a first optically transparent layer (a) on top of a second optically transparent layer (b) which has a lower refractive index than layer (a), and into which an optical grating has been inserted, which grating is oriented perpendicularly to the path of an excitation light which is coupled into the thin-film waveguide by means of said optical grating. Detection reagents are immobilized on the surface of the thin-film waveguide by way of applying in rows of spatially separated measurement areas, a mycotoxin assay (immunoassay) in the form of a substance library of immobilized binding partners for mycotoxins and/or for mycotoxin probes, and an independent control assay comprising an immobilized referencing antigen. The arrays are applied to the PWG biochip in such a way that the measurement areas are oriented in rows parallel to the optical grating. A row of the control assay is located, in the direction of the excitation light, above and below each row of immunoassay (see FIG. 1) so as to enable a referenced fluorescence intensity of the mycotoxin assay measurement area to be obtained by dividing the fluorescence intensity of the mycotoxin assay measurement area by the average of the fluorescence intensities of the control assay measurement areas adjacent in the direction of the excitation light.

Surprisingly, referencing of the immunoassays in the fluidic system turned out to be considerably improved by using the dynamic referencing concept of the invention rather than the known static referencing concept. Advantageously, dynamic referencing can compensate for both fluctuations in the fluidic system (for example adsorption in the channels, volume fluctuations, variations of the amount of antibodies in the pad) and fluctuations on the PWG biochip surface (for example attenuation, variations in spotting).

A first subject matter of the present invention is therefore a cartridge for the verification and quantitative analysis of analytes in a sample fluid, comprising a structured body into which cavities connected to one another by channels have been inserted, said cartridge having at least one inlet for introducing the analyte-containing sample fluid, at least one reagent chamber and at least one detection chamber, wherein
a. the reagent chamber accommodates, in a dry form, one or more labeled analyte probes to react with the analytes from the sample fluid and one or more labeled referencing probes to react with a referencing antigen,
b. the bottom of the detection chamber is a thin-film waveguide comprising a first optically transparent layer (a) on top of a second optically transparent layer (b) which has a lower refractive index than layer (a), with an optical grating being inserted into the layer (a) or (b), which is oriented perpendicularly to the path of an excitation light which is coupled into the thin-film waveguide by means of said optical grating,
c. an immunoassay in the form of a substance library of binding partners for analytes and/or for analyte probes, which binding partners have been immobilized in rows of spatially separated measurement areas, and an independent control assay comprising the referencing antigen immobilized in rows of spatially separated measurement areas have been applied to the surface of said thin-film waveguide, and
d. the particular row are oriented parallel to the optical grating and a row of control assays is located, in the direction of the excitation light, above and below each row of the immunoassay.

Preference is given to the control assay being selected such that the referencing antigen has a molecular weight similar to the analyte, and the referencing probe has binding properties similar to the analyte probes (affinity, binding kinetics). Moreover, the control assay must not exhibit any cross reactivity with the immunoassays, and the antigen must not naturally occur in the matrix tested.

It is furthermore advantageous for the degradation behaviors of the control assay and the immunoassay to be similar so as to provide long-term stability of the calibration curve of a production batch.

In a particular embodiment of the invention, the analytes are mycotoxins.

Preference is given to using an immunoassay as described in WO 2007/079893, the contents of which are incorporated by reference.

A preferred immunoassay comprises rows of mycotoxin-protein conjugates for example mycotoxin-BSA conjugates.

Examples of control assays are assays for mycotoxins which do not occur naturally in the matrix tested. The control assay is preferably selected such that a molecule 1000 g/mol is detected. Particular preference is given to applying to the PWG biochip a control assay for fluorescein and a row of control-protein conjugates, for example fluorescein-BSA.

The PWG biochip consists of, for example, a glass support coated with a layer of tantalum pentoxide. The layer has a thickness of from 40 to 160 nm, preferably 80 to 160 nm, particularly preferably 120 to 160 nm, very particularly preferably 155 nm. The glass support contains an optical grating with a grating depth of from 3 to 60 nm, preferably 5 to 30 nm, particularly preferably 10 to 25 nm, very particularly preferably 18 nm, and a grating period of from 200 to 1000 nm, preferably 220 to 500 nm, particularly preferably 318 nm. Preferably, the grating has a single period, i.e. it is monodiffractive.

The tantalum pentoxide surface is usually coated with dodecyl phosphate in the form of a monolayer. Analyte-protein conjugates, preferably mycotoxin-BSA conjugates, and referencing antigen-protein conjugates preferably fluorescein-BSA conjugates are immobilized on this surface. Immobilization usually comprises applying to said surface and adsorbing there the protein conjugates at concentrations of from 0.1 to 5 mg/ml, preferably 0.2 to 2 mg/ml, particularly preferably 0.5 to 1.5 mg/ml, very particularly preferably 1 mg/ml.

The protein conjugates can be applied using one or more methods selected from the following group: inkjet spotting, mechanical spotting by pin or pen, microcontact printing, fluidic contacting of the measurement areas with the biological or biochemical or synthetic recognition elements by supplying the latter in parallel or crossed microchannels, with exposure to pressure differences or to electric or electromagnetic potentials.

The areas of the PWG chip surface which are still free after immobilization of the protein conjugates are passivated by treatment with BSA in order to suppress unspecific binding.

The PWG biochip constitutes the bottom of the detection chamber of the cartridge of the invention and is integrated into said cartridge.

The cartridge consists of a structured body into which chambers and channels are inserted, with the chambers being inserted in the body preferably in such a way that they are formed at least on one side by applying a sealing unit. The structured body is sealed at the top and the bottom by means of a sealing unit, apart from the inlet, the bottom of the detection chamber and optional vents. Preference is given to positioning the biochip before the sealing unit which holds the biochip in place. The sealing unit is preferably a sealing film.

Preference is given to transporting in the channels and in the chambers a precisely defined volume of sample fluid, and this is facilitated by the design of the channels and the chamber and by employing a suitable means for transporting the sample fluid. Reaction times can likewise be precisely controlled here, improving the reproducibility of the analysis. A matching design of the chamber and the channels ensures an optimal flow profile with a reduced void volume and, where appropriate, optimal contact with the immobilized detection reagents.

The channels connect the inlet, the reagent chamber and the detection chamber to one another and usually have a diameter of from 0.1 to 2.5 mm, preferably 0.5 to 1.5 mm, particularly preferably 1 mm.

In a particular embodiment of the cartridge, the reagent chamber has a reagent pad which accommodates the analyte probes and referencing probes, in particular antibodies for mycotoxins and fluorescein.

The reagent pad is selected so as to meet the requirements of the detection chamber with regard to the required liquid volume of the supernatant solution and the concentration of the individual components in said solution.

The reagent pad usually consists of a fibrous or porous material, for example fine particles or tissue, into which reagents have been incorporated (by adsorption thereto, fixing thereto, dispersion therein, drying thereinto). A preferred reagent pad consists of glass or polymers such as, for example, cellulose. For example, reagent pads are used which are also used in lateral flow assays and which are commercially available in various forms.

A preferred reagent chamber requires a liquid volume of from 10 to 100 μl, preferably 20 to 60 μl, particularly preferably 40 μl, and analyte probes and referencing probes dissolved therein at a concentration of from 10⁻⁷ M to 10⁻¹⁰ M, preferably nanomolar concentrations.

This reagent chamber is filled by selecting the reagent pad which preferably consists of extra thick glass filters from Pall Corporation (pore size 1 μm, typical thickness 1270 μm (50 mils), typical water flow rate 210 ml/min/cm2 at 30 kPa), with two circular filter pieces with a suitable diameter (usually from 5 to 10 mm) being stacked on top of each other. The resulting reagent pad is usually impregnated with approx. 100 μl of the solution containing the fluorescently labeled probes and usually further components for supporting said impregnation. Impregnation is carried out, for example, by way of drying or lyophilization.

The reagent pad is usually operated in the cartridge in such a way that it is wetted with approx. 80 μl of sample fluid (e.g. mycotoxin extract).

After a preincubation time of from 1 to 10 min, usually from 20 to 60 μl of the solution are transported into the detection chamber.

A precise control of the volumes is advantageous in the present invention but is not necessary, since variations between the different cartridges can be compensated for by the referencing method of the invention.

The present invention also relates to a method for the verification of analytes, in particular mycotoxins, by means of the cartridge of the invention.

The second subject matter of the present invention is a method for the quantitative analysis of analytes which comprises the steps of:
a. optionally extracting the analytes from a matrix into a sample fluid,
b. carrying out the assay in the cartridge as claimed in any of claims 1 to 7, wherein, after the sample fluid has been introduced into the cartridge, the said sample fluid is transported into the reagent chamber and mixes or reacts with labeled probes applied there, then
c. transporting the sample fluid into the detection chamber and reacting the analytes and/or the labeled probe with the immunoassay and control assay, followed by
d. illuminating the thin-film waveguide to excite the labeled probes of the immunoassay and control assay for fluorescence and taking a fluorescent image, then
e. calculating the referenced fluorescence intensities of the immunoassay on the basis of the control assay, wherein the referenced fluorescence intensity of each immunoassay measurement area is calculated by dividing the fluorescence intensity of said immunoassay measurement area by the average of the fluorescence intensities of the control assay measurement areas adjacent in the direction of the excitation light, and
f. calculating and displaying the analyte data based on a calibration curve.

If the mycotoxins are present in a solid matrix, the latter is normally crushed in an optional first step of the method according to the invention, followed by extracting the mycotoxins with a suitable solvent from the matrix. Examples of extractants are aqueous solutions of methanol, ethanol or acetonitrile. Examples of solid matrices are wheat, corn, barley, rye, peanuts, hazelnuts, etc. If the extract contains more than 10% of the nonaqueous solvent, then normally a dilution step is required before the cartridge is filled. Liquid matrices (milk, fruit juice, wine, etc.) can be added to the cartridge directly or after suitable dilution.

In a further step, the user adds the extract or the sample solution to the cartridge and seals the cartridge. The cartridge is then inserted into a reader. The reader contains a pump which pumps air into the cartridge and thus transports the solution from the sample inlet into the reaction chamber, where said solution wets the reagent pad applied there.

When the reagent pad is wetted, the antibodies are removed from the reagent pad with the aid of the extract and thus mixed with said extract.

The incubation time of the extract in the reagent pad is preferably from 1 to 20 min, particularly preferably 3 to 7 min. The pump now once again pumps air into the cartridge and thereby moves the liquid volume into the detection chamber above the PWG biochip. Again an incubation step is carried out which usually lasts from 1 to 100 min, preferably 5 to 15 min.

Preferably, the cartridge is heated to a temperature which is preferably from 20 to 37° C., particularly preferably 25° C. for the duration of the method.

Incubation of the labeled antibodies on the PWG biochip is followed by coupling a laser beam into the optical grating. Excitation due to the areal illumination of the PWG biochip causes the labeled antibodies to fluoresce. The fluorescence image of the biochip is recorded with the aid of a camera and a suitable fluorescence filter.

Image analysis software which is installed on the computer of the reader then determines the fluorescence intensity of the mycotoxin and control assay measurement areas. A referenced fluorescence intensity of the mycotoxin assay measurement area is obtained by dividing the fluorescence intensity of the mycotoxin assay measurement area by the average of the fluorescence intensities of the control assay measurement areas adjacent in the direction of the excitation light. The quantitative relationship between the referenced fluorescence intensities of the mycotoxin assay measurement areas and the concentration of a mycotoxin in the solution pipetted into the cartridge is usually established by recording calibration curves. The resulting mathematical relationships are stored on the computer of the reader.

When a sample is measured, the referenced fluorescence intensity is determined after the fluorescence image has been recorded, and the corresponding mycotoxin concentration is calculated based on the calibration curve. The mycotoxin data is then displayed on the screen of the reader.

The device of the invention and the method of the invention will be illustrated in more detail on the basis of the following examples and drawings, without being limited thereto.

### BRIEF DESCRIPTION OF THE DRAWINGS

FIG. 1: Construction of the mycotoxin array

FIG. 2: Cartridge design

FIG. 3: PWG biochip side view

FIG. 4: PWG biochip dimensions

### REFERENCE NUMBERS

1 Cartridge

2 Inlet

3 Channel

4 Reagent chamber with reagent pad

5 Detection chamber

6 PWG biochip

7 Grating

8 Glass plate

9 Wave-guiding layer

10 Monolayer of dodecyl phosphate/adhesion-promoting layer

11 Reference spots/control assay

12 Mycotoxin-BSA conjugate spots/immunoassay

13 Reference spots/control assay

14 BSA

The cartridge (1) consists of a structured body into which channels and cavities have been introduced.

For example, the cartridge of the invention was produced by injection molding. The body consists of a plate made of black polyoxymethylene (POM), in which the channels and chambers have been drilled out and milled off.

The cartridge (1) comprises an inlet (2) for adding a sample fluid containing the analytes to be detected to a sample chamber of the cartridge (1), a reagent chamber with a reagent pad (4), into which the sample fluid is transported via a channel (3), and a detection chamber (5) into which the sample fluid is transported via another channel (3) and which comprises a PWG biochip (6).

The reaction chamber (4) contained antibodies labeled with a fluorescent dye which are specific for mycotoxins from the sample fluid, and labeled antibodies which are specific for fluorescein, impregnated on the reagent pad.

Both the PWG biochip (6) and the reagent pad were held between two polyolefin films in the POM plate, which films also served as sealing films for sealing the test cassette. The upper sealing film had a thickness of 180 μm and the lower sealing film had a thickness of 80 μm.

The lower film had in the region of the PWG biochip (6) a window which provided free access to the measurement region of the PWG biochip (6).

At the start of the assay, the sample fluid was introduced through the inlet (2) into the sample chamber, and the inlet (2) was provided with an airtight seal by way of a suitable lid. A defined volume of air was introduced into the cartridge (1) at the inlet with the aid of the transport unit. This volume of air displaced the sample fluid which therefore entered the reagent chamber (4) and completely wetted the reagent pad.

Due to the reagent chamber (4) being charged with the sample fluid, the antibodies were dissolved, mixed with the sample fluid and formed a specific bond with the mycotoxins present in said sample fluid (mycotoxin-antibody conjugate). With the amount of mycotoxins in the sample fluid increasing, the free binding sites of the antibodies became increasingly saturated.

After a certain dwell time (10 minutes) at a temperature of 25° C., the sample fluid containing mycotoxin-antibody conjugates and the antibodies for fluorescein was transported into the detection chamber (5) in a next step.

In the detection chamber (5), the course or the endpoint of the biochemical detection reaction were detected.

The detection chamber (5) was filled completely with the sample fluid. The entire channel system was ventilated. Ventilation of the complete channel system was carried out through ventilation openings applied to the upper sealing film.

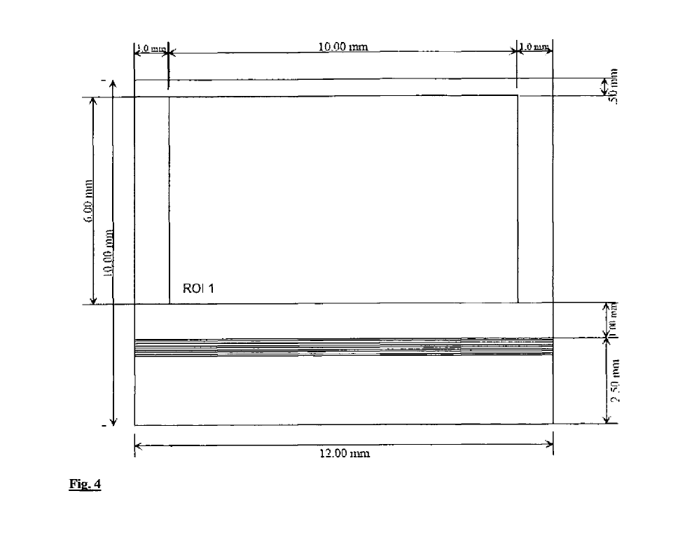

The detection chamber (5) comprised a PWG biochip (6). FIG. 2 depicts a top view diagram of the PWG biochip (6), and FIG. 3 represents a side view diagram of the PWG biochip (6).

The PWG biochip (6) in the detection chamber (5) consisted of a 10 mm×12 mm glass plate (8) of 0.7 mm (12.0+/−0.05 mm ×10.0+/−0.05 mm×0.70+/−0.05 mm) in thickness. A thin, 155 nm wave-guiding layer (9) of Ta₂O₅ (tantalum pentoxide) was located on one side of the PWG Chip (6). The measurement region of the chip consisted of a central 10 mm×6 mm rectangular area. Parallel to this measurement region, there is a crescent-shaped band of 500 μm in width: the grating (7) for coupling of the excitation light. The accuracy of the position of the grating (7) with respect to the edges was +/−0.05 mm. The grating depth was 18 nm and the grating period was 318 nm with a duty cycle of 0.5.

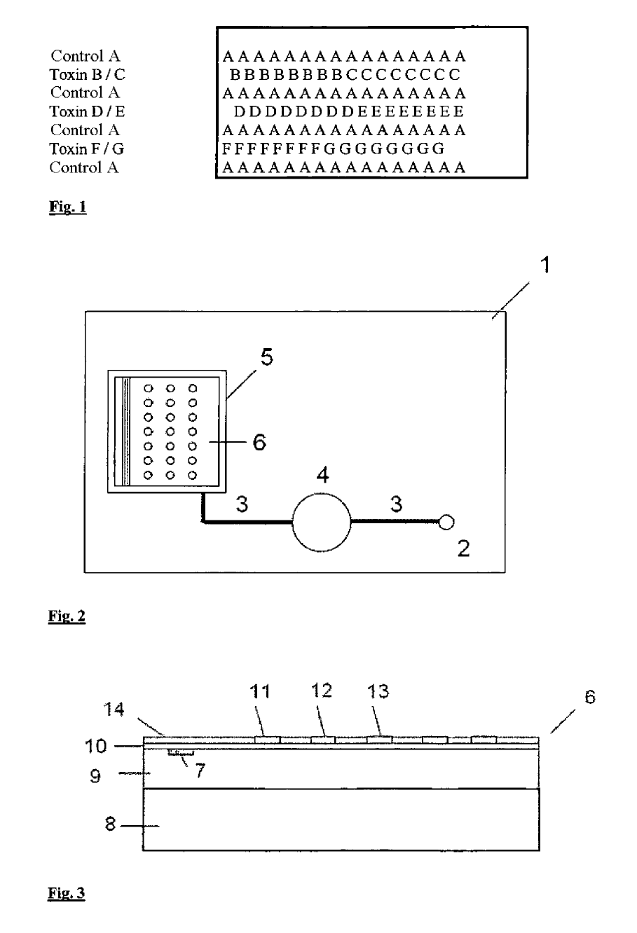

A monolayer of dodecyl phosphate was applied as adhesion-promoting layer (10) to the PWG biochip (6). The adhesion-promoting layer (10) contained mycotoxin-BSA conjugates applied dropwise/immobilized thereto in an adsorptive manner in the form of an immunoassay (12) in the form of rows of spots parallel to the optical grating (arrays). Above and below each row of mycotoxin-BSA conjugate spots (immunoassay (12)) there was a row of BSA-fluorescein spots (control assay/reference spots (11, 13)) (FIG. 1). The free areas between the immunoassays (12) and control assays was blocked with BSA (14) (passivation).

In the detection chamber (5), the mycotoxin-antibody conjugate and, where appropriate, antibodies with free binding sites and also the antibodies for fluorescein reach the immunoassay (12) of immobilized analyte-BSA conjugates and, respectively, the control assay (11, 13) on the PWG biochip (6). Antibodies with free binding sites formed a specific bond with the corresponding immobilized analyte-BSA conjugates.

The more antibodies with free binding sites were present in the solution, i.e. the lower the proportion of the corresponding analytes in the sample fluid, the more antibodies labeled with a fluorescent dye were bound to the PWG biochip. The antibodies saturated with analytes in the sample fluid remained in the solution.

By coupling electromagnetic radiation into the PWG biochip (6), it was possible to excite the antibodies bound to the immobilized analyte-BSA conjugates and labeled with a fluorescent dye to fluoresce in the evanescent field of the waveguide. The antibodies labeled with a fluorescent dye that were in solution were not excited in this case. In this way, the mycotoxins present in the sample fluid were indirectly quantified.

A referenced fluorescence intensity of the mycotoxin spot was obtained by dividing the fluorescence intensity of the mycotoxin spot by the average of the fluorescence intensities of the reference spots.

The quantitative relationship between the referenced fluorescence intensities of the mycotoxin spots and the concentration of a mycotoxin in the solution pipetted into the cartridge was established by recording calibration curves. The resulting mathematical relations were stored on the computer of the reader.

### Example 1

### Preparation of Cartridges for Determining Deoxynivalenol (DON) on a PWG Biochip

Twenty-four PWG biochips (Unaxis, Liechtenstein), outer dimensions: 10 mm×12 mm, made of glass and provided with a layer (155 nm) of tantalum pentoxide into which an optical grating (grating depth 18 nm) had been imprinted, were purified and coated with dodecyl phosphate. Conjugates of deoxynivalenol and bovine serum albumin (DON-BSA, Biopure, Austria) and conjugates of bovine serum albumin and fluorescein (BSA-FITC, Sigma, Germany) were applied to the biochip with the aid of a spotter of the Nanoplotter (Ge-SIM, Germany) type. The spots were applied to the PWG biochip in the form of alternating rows of in each case 16 BSA-FITC conjugate spots and BSA-DON conjugate spots such that in each case the rows ran parallel to the optical grating. The spots were dried and then subjected to the fog of an aqueous BSA solution. The PWG biochips were washed and then dried. The PWG biochips were bonded in cartridges using double-sided adhesive tape. Said cartridges contained a sample chamber for receiving the samples, a reagent chamber with a glass fiber pad and a detection chamber for the PWG biochip. The chambers were connected to one another by channels. The glass fiber material was impregnated with solutions of nanomolar concentrations of antibodies labeled with the fluorescent dye DY-647 (Dyomics, Germany), using monoclonal antibodies to deoxynivalenol and fluorescein. The antibodies had been dissolved in a buffer containing BPS (=phosphate buffered saline), 0.1% ovalbumin, 0.05% Tween and 5% sucrose. The reagent pads obtained were dried in vacuo and then printed into the cartridges. The cartridges were sealed on both sides with sealing films in order to seal the channels.

### Example 2

### Recording a Standard Curve (Calibration Curve) for Quantification of DON

Solutions of DON at concentrations ranging from 0 to 6000 ppb were prepared, and 17 separate cartridges were charged in each case with 200 μl of said solution. The cartridges were sealed and then inserted into the MyToLab reader (Bayer Technology Services, Germany). The reader was set such that the internal transport unit of the instrument transported the fluid inserted into the cartridge first into the reagent pad and, after 5 minutes of preincubation time, into the detection chamber. The temperature was maintained at 25° C. throughout. After 10 min of incubation time in the chip chamber, the laser was coupled into the optical grating of the PWG biochip. A fluorescence image of each individual PWG biochip was recorded with an integration time of 2 to 3 s. The fluorescence intensities obtained for each DON spot were divided by the average of the fluorescence intensities of the BSA-FITC spots located above and below the particular DON spot. The averages of the fluorescence intensities of all 16 DON spots referenced in this way were determined. The concentration-dependent, referenced fluorescence intensities obtained were fitted by a sigmoidal fit with the aid of the computer program Origin 7G (Origin Lab Corporation, USA).

### Example 3

### Measurement of DON in Artificially Contaminated Wheat Samples

Wheat grains were ground, and the resulting flour was treated with a known amount of a DON solution which was left to dry. The homogenized sample contained 888 mg/kg (ppb) DON. Five g of the flour sample were extracted with 25 ml of 70% methanol by vigorously shaking for 3 min. The extract was left to settle, and the supernatant was diluted with buffer in a 1:3 ratio. The diluted extract was added to 7 different cartridges. The cartridges were then measured, as described above, in the MyToLab reader and the referenced fluorescence intensities of the DON spots were determined. The DON concentrations in ppb were determined in relation to the above-described standard curve, producing values of 1042, 757, 710, 660, 431, 728 and 984 ppb. The average of the DON determination was 760 ppb with 27% standard deviation.

1. A cartridge for the verification and quantitative analysis of analytes in a sample fluid, comprising a structured body into which cavities connected to one another by channels have been inserted, said cartridge having at least one inlet for introducing the analyte-containing sample fluid, at least one reagent chamber and at least one detection chamber, wherein
a. the reagent chamber accommodates, in a dry form, one or more labeled analyte probes to react with the analytes from the sample fluid and one or more labeled referencing probes to react with a referencing antigen,
b. the bottom of the detection chamber is a thin-film waveguide comprising a first optically transparent layer (a) on top of a second optically transparent layer (b) which has a lower refractive index than layer (a), with an optical grating being inserted into the layer (a) or (b), which is oriented perpendicularly to the path of an excitation light which is coupled into the thin-film waveguide by means of said optical grating,
c. an immunoassay in the form of a substance library of binding partners for analytes and/or for analyte probes, which binding partners have been immobilized in rows of spatially separated measurement areas, and an independent control assay comprising the referencing antigen immobilized in rows of spatially separated measurement areas have been applied to the surface of said thin-film waveguide, and
d. the particular row are oriented parallel to the optical grating and a row of control assays is located, in the direction of the excitation light, above and below each row of the immunoassay.

2. The cartridge as claimed in claim 1, wherein the referencing antigen has a molecular weight similar to that of the analyte, the referencing probe has binding properties similar to the analyte probes, the control assay does not exhibit any cross reactivity with the immunoassays, and the referencing antigen is not present in the matrix tested.

3. The cartridge as claimed in claim 1, wherein the analyte probes are antibodies.

4. The cartridge as claimed in claim 1, wherein the analytes are mycotoxins.

5. The cartridge as claimed in claim 4, wherein the referencing antigen is 1000 g/mol in the control assay.

6. The cartridge as claimed in claim 4, wherein the referencing antigen is fluorescein.

7. The cartridge as claimed in claim 4, wherein the immunoassay includes mycotoxin-protein conjugates and/or the control assay includes control molecule-protein conjugates.

8. A method for the quantitative analysis of analytes, comprising the steps of:
a. optionally extracting the analytes from a matrix into a sample fluid,
b. carrying out the assay in the cartridge as claimed in claim 1, wherein, after the sample fluid has been introduced into the cartridge, the said sample fluid is transported into the reagent chamber and mixes or reacts with labeled probes applied there, then
c. transporting the sample fluid into the detection chamber and reacting the analytes and/or the labeled probe with the immunoassay and control assay, followed by
d. illuminating the thin-film waveguide to excite the labeled probes of the immunoassay and control assay for fluorescence and taking a fluorescent image, then
e. calculating the referenced fluorescence intensities of the immunoassay on the basis of the control assay, wherein the referenced fluorescence intensity of each immunoassay measurement area is calculated by dividing the fluorescence intensity of said immunoassay measurement area by the average of the fluorescence intensities of the control assay measurement areas adjacent in the direction of the excitation light, and
f. calculating and displaying the analyte data based on a calibration curve.

9. The method as claimed in claim 8, in which the cartridge is heated to a temperature of from 20 to 37° C. for the duration of the method.

10. The method as claimed in claim 8, wherein the reaction in step b. takes from 1 to 20 min and/or the reaction in step c. takes from 1 to 100 min.

11. A method for the verification and quantitative analysis of mycotoxins comprising carrying out the method in the cartridge as claimed in claim 1.
